(19) United States
(12) Patent Application Publication
(10) Pub. No.: US 2015/0250750 A1
(43) Pub. Date: Sep. 10, 2015

(54) PHARMACEUTICAL COMPOSTIONS OF DICLOFENAC OR SALTS THEREOF

(71) Applicant: WOCKHARDT LIMITED, Aurangabad (IN)

(72) Inventors: Moinuddin Syed, Aurangabad 1 (IN); Nitin Martandrao Mehetre, Buldhana 3 (IN); Rahul Sudhakar Dabre, Nagpur 5 (IN); Girish Kumar Jain, Delhi 4 (IN)

(21) Appl. No.: 14/430,939

(22) PCT Filed: May 3, 2013
(86) PCT No.: PCT/IB2013/053515

§ 371 Date: Mar. 25, 2015

(30) Foreign Application Priority Data
Oct. 16, 2012 (IN) 3028/MUM/2012

(51) Int. Cl. A61K 31/196; A61K 47/14; A61K 47/22; A61K 9/48; A61K 47/10

(52) U.S. Cl. CPC A61K 31/196; A61K 9/4825; A61K 47/10; A61K 47/22; A61K 47/14

(57) ABSTRACT

The present invention refers to an improved pharmaceutical composition of diclofenac or pharmaceutically acceptable salt and pharmaceutically acceptable excipients wherein the composition is substantially free of dispersing agents. By judicially using pharmaceutical excipients other than dispersing agent, pharmaceutical composition of diclofenac or its salts with rapid and uniform gastrointestinal absorption of diclofenac can be achieved. The composition of the invention can also minimize the controllable side effects of diclofenac.

### FIELD OF INVENTION

The present invention relates to improved pharmaceutical compositions of diclofenac or pharmaceutically acceptable salt and pharmaceutically acceptable excipients wherein the composition is substantially free of dispersing agents. By judicially using pharmaceutical excipients other than dispersing agent, pharmaceutical composition of diclofenac or its salts with rapid and uniform gastrointestinal absorption of diclofenac can be achieved. The composition of the invention can also minimize the controllable side effects of diclofenac.

### BACKGROUND OF THE INVENTION

Within the pharmaceutical art, the formulation of pharmaceutically active compounds into usable dosage forms, in which the absorption of the active ingredient is optimized and the extent of controllable side effects is minimized, is challenging to pharmaceutical formulation scientists and, frequently, unpredictable. Representatives of these compounds include, for example, pharmaceutical agents well known in the art as non-steroidal anti-inflammatory drugs (NSAIDs) e.g. Diclofenac.

Diclofenac is one of the routinely prescribed anti-inflammatory agents available for the management of pain and inflammation. It is marketed as injection, oral immediate release tablets, sustained release tablets, liquid filled capsules and conventional topical formulations. The drug is almost completely absorbed after oral administration.

A major portion of commercial diclofenac is available in the form of oral medications. It is widely known that the drug causes serious adverse effects in the gastrointestinal tract. Gastrointestinal bleeding and ulcerations are quite common due to oral diclofenac.

U.S. Pat. No. 4,880,835 discloses oral liquid compositions of calcium sulindac with a pharmaceutical vehicle using glycol, a polyol, and alcohol.

K. Chan, et al., Pharma Research, 7:1027 (1990) discloses that diclofenac sodium in form of aqueous solution posses less bioavailability in comparison to its enteric coated tablet.

U.S. Pat. No. 4,704,405 discloes that NSAIDs, such a sulindac has absorption problem from the gastrointestinal tract when administered orally.

U.S. Pat. No. 5,183,829 discloses oral liquid NSAID formulation containing dispersing agents. The formulation was found to be unsuitable for filling in soft gelatin capsules, as it become tacky due to the inside composition, and adhere to adjacent soft capsules.

U.S. Pat. No. 6,365,180 descloses liquid and semi-solid compositions of NSAIDs containing dispersing agents.

Thus, several methods and compositions of diclofenac have been taught in the art using dispersing agents. Despite this, these compositions may either be unsuitable to present in particular product form, or may provide uncertain control over the rate of absorption of the active ingrdient or on the side effects.

Further, on administering the oral solution of diclofenac, it mixes with stomach acid, can form agglomerates, which sediments in a brief period of time over gastrointestinal pasage, making diclofenac less biologically available, and thus exhibit poor gastrointestinal absorption. Prior art references indicates using dispensing agents in the composition in order to inhibit agglomertion.

Hence, there exists an enduring need for alternate, improved and stable pharmaceutical composition of diclofenac or its salts, which can exhbit rapid and uniform gastrointestinal absorption of diclofenac and simultaneously, without compromising on the product stability. Further, the composition ought to minimize the controllable side effects of diclofenac.

### SUMMARY OF THE INVENTION

In one aspect, the present invention provides a pharmaceutical composition of diclofenac or pharmaceutically acceptable salt thereof comprises one or more pharmaceutically acceptable excipients; wherein the composition is substantially free of any dispersing agent.

In another aspect, the present invention provides a pharmaceutical composition of diclofenac or pharmaceutically acceptable salt thereof comprises one or more solubilizing agents, one or more surfactants, and one or more plasticizing agents; wherein the composition is substantially free of dispersing agent.

In another aspect, the present invention provides a pharmaceutical composition of diclofenac or pharmaceutically acceptable salt thereof comprises one or more pharmaceutically acceptable excipients; wherein the composition is substantially free of dispersing agent, and characterized in that the composition comprises from about 5% to about 90% w/w of surfactant.

In another aspect, the present invention provides a pharmaceutical composition of diclofenac or pharmaceutically acceptable salt thereof comprises one or more pharmaceutically acceptable excipients; wherein the composition is substantially free of dispersing agent, and characterized in that the composition exhibits no significant difference in one or both of the rate and extent of absorption of diclofenac or salts thereof as compared to formulation of diclofenac marketed under the trade name Zipsor®.

In another aspect, the present invention provides a pharmaceutical composition of diclofenac or pharmaceutically acceptable salt thereof comprises one or more pharmaceutically acceptable excipients; wherein the composition is substantially free of dispersing agent, and characterized in that said composition retains at least 90% w/w of total potency of diclofenac or salt thereof after storage at 40° C. and 75% relative humidity for at least 3 months.

In another aspect, the present invention provides a dosage form selected from liquid, soft gelatin capsule, or hard gelatin capsule comprising diclofenac or pharmaceutically acceptable salt thereof and one or more pharmaceutically acceptable excipients; wherein the composition is substantially free of any dispersing agent.

In another aspect, the present invention provides a method of improving the rate of absorption of diclofenac or pharmaceutically acceptable salt thereof in the patient, comprising administering to the patient in need of the treatment with diclofenac a composition comprising diclofenac or pharmaceutically acceptable salt thereof and one or more pharmaceutically acceptable excipients; wherein the composition is substantially free of dispersing agent.

In another aspect, the present invention provides a method of accelerating the onset of the therapeutic benefit provided by diclofenac or pharmaceutically acceptable salt thereof in the patient, comprising administering to the patient in need of the treatment with diclofenac a composition comprising diclofenac or pharmaceutically acceptable salt thereof and one or more pharmaceutically acceptable excipients; wherein the composition is substantially free of dispersing agent.

In another aspect, the present invention provides use of a pharmaceutical composition for the preparation of medicaments useful for providing relief from mild to moderate acute pain, the composition comprising of diclofenac or pharmaceutically acceptable salt thereof and one or more pharmaceutically acceptable excipients; wherein the composition is substantially free of dispersing agent.

Embodiments of the pharmaceutical composition may include one or more of the following features. For example, the pharmaceutically acceptable excipients may include diluents, disintegrants, binders, bulking agents, anti-adherents, anti-oxidants, buffering agents, colorants, flavoring agents, coating agents, plasticizers, stabilizers, preservatives, lubricants, glidants, chelating agents, and the like known to the art used either alone or in combination thereof.

### DETAILED DESCRIPTION OF THE INVENTION

The present invention provides a solution to the aforesaid shortcomings. Particularly, the invention provides pharmaceutical formulations of diclofenac or salt thereof which is substantially free of any dispersing agent.

The inventors of the present invention have surprisingly found that it is possible to devise diclofenac compositions with improved oral bioavailability without using any dispersing agent. The inventors of the present invention further empirically found that when pharmaceutical excipients other than dispersing agents are judicially used, the resulting composition can exhibit rapid and uniform gastrointestinal absorption of diclofenac and simultaneously, that to without compromising on the product stability. Further, the composition advantageously may minimize the controllable side effects of diclofenac.

The present invention relates to a novel pharmaceutical composition of diclofenac or salt thereof for oral administration, and methods of using such compositions for enhancing the rate and degree of absorption of diclofenac or salt thereof from such compositions, and for minimizing gastric irritation induced or caused by ingestion of diclofenac or salt thereof.

The marketed formulations of diclofenac posses limited flexibity of formulationg in the form different dosage form. For instance, the liquid composition of diclofenac in marketed product, Zipsor® (marketed in USA by Depomed Inc.) is unsuitable to fill in hard gelatin capsules. Other formulations suggested in the prior art unsuitable for filling in soft gelatin capsules, as it become tacky due to the inside composition, and adhere to adjacent soft capsules.

The compositions of diclofenac or salt thereof in accordance with the present invention posses excellent storage stability and flexibility of presenting in the form of a wide range of products, such as in the form of soft gelatin capsule or hard gelatin capsule. Moreover, the inventors of the present invention have devised a diclofenac composition which is bioequivalent to its marketed formulation Zipsor®.

In an embodiment, the pharmaceutical composition of diclofenac or pharmaceutically in accordance with the present invention exhibits no significant difference in one or both of the rate and extent of absorption of diclofenac or salts thereof as compared to formulation of diclofenac marketed under the trade name Zipsor®.

The composition can be devised in the form of liquid and semi-solid compositions, which can be administered in liquid form or can be used for preparing capsules containing such pharmaceutical compositions. The composition of diclofenac in accordance with the present invention in liquid form may demonstrate good reproducible distribution in gastric juice and, thereby, better absorption.

The pharmaceutical composition of of the present invention comprising diclofenac or salt thereof and one or more pharmaceutically acceptable excipients; wherein the composition is substantially free of dispersing agent.

Preferred sals of diclofenac suitable for use in the present invention include, but not limited to sodium and potassium salt, and potassium salt being more preferred.

In particular, the composition of the present invention is substantially free of any dispersing agents known in the art. For instant, the composition is substantially free of dispersing agents used in various marketed formulation of diclofenac (e.g. Zipsor®), such as polymer or carbohydrate based dispersing agents. Polymer based dispersig agents includes polyvinylpyrrolidone, and carbohydrate based dispersing agents includes hydroxypropylmethylcellulose, hydroxypropylcellulose, and cyclodextrins.

The term “substantially free” used throughout the specification refers to pharmaceutical compositions of diclofenac or salt thereof comprising less than about 10%, preferably less than 0.1% of dispersing agent by weight of diclofenac or salt thereof.

In an embodiment, the composition in accordance with present invention is useful as oral, liquid medicaments which can also be used to fill soft or hard gelatin capsules or solidified, as taught herein, to be used in hard capsules, particularly soft gelatin capsules and hard gelatin capsules, respectively.

The compositions of the present invention comprise a pharmaceutically non-toxic and therapeutically effective amount of diclofenac or salt thereof. Accordingly, any suitable non-toxic and therapeutically effective amount of diclofenac known in the art can be used.

In an embodiment, the amount of diclofenac or salt thereof in the composition of the present invention may range from about 0.1% to about 95% w/w of the composition.

In another embodiment, the amount of diclofenac or salt thereof in the composition of the present invention may range from about about 1% to about 30% w/w of the composition.

Suitable pharmaceutical excipients for use in the composition of the present invention comprise one or more of solubilizing agents, surfactants, and plasticizing agents.

The pharmaceutical composition comprises one or more pharmaceutically acceptable non-toxic solubilizing agents. Such readily available solubilizing agents are well known in the art and are typically represented by the family of compounds known as polyethylene glycols (PEG) having molecular weights from about 200 to about 8,000. For compositions of the present invention when a liquid is desired for the final formulation or a liquid is to be used to fill soft capsules, preferably soft gelatin capsules, preferred molecular weights range from about 200 to about 600 with PEG 400 being especially preferred. For composition of the present invention when a semi-solid is preferred, especially for filling a hard capsule, preferably a hard gelatin capsule, preferred molecular weight is about 3350 while an especially preferred molecular weight is 3350 plus sufficient 400 molecular weight PEG to improve capsule filling characteristics.

Another solubilizing agent, which may be utilized in compositions of the present invention, is water, preferably purified, and more preferably, deioniozed. For such compositions, the concentration of water is from about 0.01% to about 95% w/w.

In an embodiment, when the compositions of the present invention is filled into soft gelating capsules, the amount of water in the composition ranges from about 0.01% to about 5%.

In a further embodiment, when more than one plasticizing agent are used in the compositions of the present invention, the amount of solubilizing agent may range from about 0.01% to about 80%.

In a further embodiment, the preferred concentration of solubilizing agent in the compositions is from about 60% to about 90% w/w.

The pharmaceutical composition of the present invention further optionally comprises one or more non-toxic plasticizing agents. The plasticizing agents, which are well known in the pharmaceutical formulation art, include, for example, glycerin, propylene glycol, and sorbitol. Such commercially available plasticizers can be prepared to include more than one plasticizing agent component.

In an embodiment, the compositions of the present invention comprise glycerin as the preferred plasticizing agent.

In a further embodiment, propylene glycol may be used both as a plasticizing agent and as a solubilizing agent when used alone or in combination with another solubilizing agent.

The amount of plasticizing agent suitable for use in the composition of the present invention may range from about 0.1% to about 75% w/w.

In an embodiment, the amount of plasticizing agent ranges from about 0.1% to about 50% w/w. In a further embodiment, the amount of plasticizing agent ranges from about 1% to about 30% w/w.

In a further embodiment, when the compositions of the present invention is filled into soft capsules, the amount of plasticizing agent may range from about 5% to about 10% w/w.

The pharmaceutical composition of the present invention further optionally comprises one or more non-toxic surfactants selected one or more from anionic, cationic, non-ionic and zwitterionic surfactant. Non-ionic surfactant is preferred.

Examples of suitable surfactants include macro gel esters (Labrafils), Tandem 522®, Span 80®, Geluciere® such as, for example, Geluciere 44/14, tocopherol polyethylene glycol 1000 succinate; polysorbate 20; and polysorbate 80. Geluciere® is more preferred.

Surprisingly, it was found that when high amount of surfactant is used in the composition of present invention, it may render faster, reproducible, and a more uniform absorption rate of diclofenac, without using any dispersing agent.

The amount of surfactant suitable for use in the composition of the present invention may range from about 0.1% to about 95% w/w.

In an embodiment, the amount of surfactant ranges from about 5% to about 90% w/w. In a further embodiment, the amount of surfactant ranges from about 20% to about 90% w/w.

The order of addition of the various components of the present invention will not affect the formation of a solution, when desired, of the present invention. However, when surfactant is used, it may be added after the addition of diclofenac or salt thereof.

In an embodiment, the process of preparing the pharmaceutical composition of the present invention comprises the steps of:

(a) forming a mixture of one or more solubilizers by heating;
(b) adding one or more surfactants to the heated mixture of solubilizers formed in step (a);
(c) adding diclofenac or salt thereof to the mixture formed in step (b) by heating to form a liquid; and
(d) optionally, filling the liquid formed in step (c) in soft or hard gelating capsules.

The mixture of polyethylene glycol 400, propylene glycol and water was heated with stirring. Gelucire and Vitamin E TPGS were added to the heated mixture, and the heating is continued with stirring until the Gelucire and Vitamin E TPGS were completely dissolved. Diclofenac potassium was then added to the heated mixture with stirring until diclofenac potassium was completely dissolved. The mixture was allowed to cool to ambient temperature and then filled into hard gelatin capsules using standard procedures

It was found that the aforesaid process of preparing the pharmaceutical composition may provide the surprising result of maintaining diclofenac in solution during the process, resulting in a stable pharmaceutical composition of the present invention with its attending benefits as set forth herein.

The capsules filled with pharmaceutical composition of the present invention may be coated with any non-toxic, pharmaceutically acceptable coating. Such coatings include, for example, enteric, taste-masking, color-coating, sustained or delayed release, non-performance flavor coatings, and the like, and are prepared and applied via techniques well known to one of ordinary skill in the art.

Other pharmaceutically acceptable, non-toxic pharmaceutical additives may be included in the compositions of the present invention and include, for example, sweetening agents, local anesthetics, antibacterials, a lower alkyl alcohol such as ethanol, and the like.

Accordingly, the novel compositions of the present invention provide beneficial pharmaceutical properties while being substantially free of dispersing agtent and utilizing a minimum number of components.

Diclofenac is known to cause gastrointestinal irritation, typically in the form of peptic ulceration, bleeding, and perforation. Because of the improved absorption or bioavailability without using any dispersing agent, such composition may inhibit side effects of diclofenac, such as gastroirritation induced by chronic use of diclofenac.

As used herein, the term “inhibit” is defined to include its generally accepted meaning and includes, without limitation, a reduction, holding in abeyance, and/or minimizing the side effects (e.g. gastroirritation) induced and/or resulting from the administration of diclofenac compared to such side effects (e.g. gastroirritation) induced and/or resulting from the administration of conventional pharmaceutical formulations of diclofenac.

The present invention further provides a method of improving the rate of absorption of diclofenac or salt thereof in patients, comprising administering the composition of the present invention to a patient in need of the treatment of diclofenac.

The present invention further provides a method of accelerating the onset of the therapeutic benefits of diclofenac or salt thereof in patients, provided by diclofenac comprising administering the composition of the present invention to a patient in need of the treatment with diclofenac or salt thereof.

The composition of the present invention may be formulated to deliver a typical, non-toxic daily dosage level of from about 0.25 mg to about 400 mg per day of diclofenac. Preferred doses diclofenac used in the composition of the present invention will, of course, be determined by the particular circumstances surrounding the case including, for example, an attending physician considering the state of being of the patient and the severity of the pathological condition being treated. Preferred daily doses may range from about 10 mg to about 2,000 mg per day. Typically, the composition of the present invention may be formulated to deliver about 10 mg to 500 mg per teaspoon of a liquid product.

The liquid or semi-solid composition of the present invention can be used to fill capsules, particularly hard gelatin capsules and, especially, soft gelatin capsules wherein the amount of diclofenac in each such capsule may range from about 10 mg to about 250 mg.

The present invention further provides a method of treating paroxysmal headaches, particularly migraine headaches comprising administering to a patient, in need of such treatment, a composition of the present invention comprising diclofenac or salt thereof, preferably in capsule form, and especially in hard gelatin capsule form.

Furthermore, composition of the present invention in which diclofenac or salt thereof, preferably administered in combination with, concurrent to, or subsequent to the administration of a motility agent as taught above, provides more rapid relief from pain, as a general analgesic, and particularly from injury or from surgical procedures such a dental surgery, hysterectomy, and arthroscopy.

In addition to the analgesic effect, such composition also provides more rapid relief from inflammation caused by injury, stress, surgical procedures, and the like. The dosage regime and dosage strength for using such compositions of the present invention for analgesic and anti-inflammation are as set forth above for the treatment of paroxysmal headache.

Accordingly, another aspect of the present invention provides a method of treating pain and for treating inflammation in a patient, comprising administering to the patient in need of treatment a composition of the present invention, preferably in capsule form, and especially in hard gelatin capsule form.

As used herein, the term “treatment”, or a derivative thereof, contemplates partial or complete inhibition of the stated disease state such as, for example, pain, when a composition of the present invention is administered prophylactically or following the onset of the disease state for which such composition of the present invention is administered.

“Bioequivalency” is established by a 90% Confidence Interval (CI) of between 0.80 and 1.25 for both Cmax and AUC under USFDA regulatory guidelines, or a 90% CI for AUC of between 0.80 to 1.25 and a 90% CI for Cmax of between 0.70 to 1.43 under the European regulatory guidelines (EMEA).

The term “confidence interval” as used herein refers to the plain meaning known to one of ordinary skill in the art. The confidence interval refers to a statistical range with a specified probability that a given parameter lies within the range.

The term “covariance” as used herein refers to the plain meaning known to one of ordinary skill in the art. It is a statistical measure of the variance of two random variables that are observed or measured in the same mean time period. This measure is equal to the product of the deviations of corresponding values of the two variables from their respective means.

The bioequivalence studies were carried out between Zipsor® (reference) and compositions of the invention (test) in fed state. The study was monitored in terms of Cₘₐₓ, AUC, Tₘₐₓ achieved with the test products and the reference product (Zipsor®).

The compositions of the invention exhibits pharmacokinetic profile characterized by Cₘₐₓ of about 330.6 to 423.5μg/ml, Tₘₐₓ of about 1.2 to 2.4h, AUC₀₋ₜ of about 830.43 to 1135.24 μg.h/ml, AUC_{inf} of about 843.76 to 1308.78 μg.h/ml.

At 90% confidence interval; area under the concentration time curve (AUC₀₋ₜ and/or AUC_{inf}) and maximum plasma concentration (Cₘₐₓ) values of composition of the invention lies between 0.70 and 1.70 as compared to that obtained by a 25 mg diclofenac potassium formulation marketed under the trade name Zipsor®.

The relative bioavailability study of the test composition and the reference formulation as demonstrated in Example 2 & 3 (Table 2 & 3) concludes that the composition explored in present invention provides equivalent rate and/or extent of absorption compared to diclofenac potassium formulation marketed under the trade name Zipsor®.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example 1

Diclofenac Potassium Liquid Composition:

TABLE 1

| Sr. No. | Ingredients | Mg/Capsule |
|---|---|---|
| 1 | Diclofenac Potassium | 25 |
| 2 | Proplyene Glycol | 21.6 |
| 3 | Polyethylene Glycol-400 | 21.6 |
| 4 | Water | 5.8 |
| 5 | Lauroyl polyoxylglycerides (Gelucire | 200 |
| | 44/14) | |
| 6 | Vitamin E TPGS (d-Tocopheryl | 126 |
| | polyethylene glycol 1000 succinate) | |
| | Total weight | 400 |

Process: The mixture of polyethylene glycol 400, propylene glycol and water was heated with stirring. Gelucire and Vitamin E TPGS were added to the heated mixture, and the heating is continued with stirring until the Gelucire and Vitamin E TPGS were completely dissolved. Diclofenac potassium was then added to the heated mixture with stirring until diclofenac potassium was completely dissolved. The mixture was allowed to cool to ambient temperature and then filled into hard gelatin capsules using standard procedures.

### Example 2

Bioequivalence data of the composition of the invention against Zipsor® with respect to pharmacokinetic parameters:

TABLE 2

| Sr. | Pharmacokinetic | | Composition of |
|---|---|---|---|
| No. | Paramaters | Zipsor ® | the Invention |
| 1 | Cₘₐₓ | 390.09 | 375.4 |
| 2 | Tₘₐₓ | 2.05 | 1.64 |
| 3 | AUC₀₋ₜ (μg · h/ml) | 793.49 | 941.34 |
| 4 | AUC_{inf} (μg · h/ml) | 974.23 | 976.67 |

### Example 3

Bioequivalence data with respect to Test (Composition of the invention) to reference Zipsor® ratios (T/R ratios) at 90% Confidence Interval (C.I.) under Fed condition:

TABLE 3

| Sr. | Pharmacokinetic | | 90% C.I. | | |
|---|---|---|---|---|---|
| No. | Paramaters | Ratio | Lower | Upper | % CV |
| 1 | LnCₘₐₓ (μg/ml) | 85.60 | 52.64 | 139.17 | 51.71 |
| 2 | LnAUC₀₋ₜ (μg · h/ml) | 112.23 | 91.36 | 20.82 | 18.48 |
| 3 | LnAUC_{inf} (μg · h/ml) | 101.86 | 78.45 | 132.26 | 26.61 |

While the invention has been described in terms of its specific embodiments, certain modifications and equivalents will be apparent to those skilled in the art and are intended to be included within the scope of the invention.

### Example 4

### Stability and Dissolution Study

The composition in accordance with the invention was subjected to stability study at 40° C. and 75% relative humidity.

TABLE 4

| Related substance | Initial | 1M 400/75 RH | 3M 400/75 RH |
|---|---|---|---|
| Diclofenac related | 0.01 | 0.042 | 0.069 |
| compound A | | | |
| Unknown at 1.37 | 0 | 0 | 0.001 |
| Unknown at 1.57 | 0.009 | 0.161 | 0.185 |
| Unknown at 1.58 | 0.061 | 0.086 | 0.11 |
| Unknown at 1.62 | 0 | 0 | 0.08 |
| Total | 0.212 | 0.446 | 0.511 |
| Assay | 99.8 | 100.8 | 97.5 |

Table 5 & 6 provides dissolution profile of marketed product Zipsor® and the composition of invention respectively when the dissolution study was performed after 1 and 3 month storage in Phosphate buffer of pH 6.8 using USP Type II dissolution apparatus and 50 rpm speed.

TABLE 5

| Time points | Diclofenac Released (%) |
|---|---|
| 10 | 13 |
| 15 | 76 |
| 20 | 100 |
| 30 | 101 |
| 45 | 100 |

TABLE 6

| | | Diclofenac Released (%) | |
|---|---|---|---|
| Time points | Initial | 1M 400/75 RH | 3M 400/75 RH |
| 10 | 35 | 29 | 50 |
| 15 | 54 | 41 | 60 |
| 20 | 67 | 47 | 68 |
| 30 | 78 | 64 | 76 |
| 45 | 89 | 85 | 88 |

Result of the stability dissolution study indicates that diclofenac composition in accordance with the present invention exhibits excellent storage stability.

1. A pharmaceutical composition of diclofenac or pharmaceutically acceptable salt thereof comprising one or more pharmaceutically acceptable excipients; wherein the composition is substantially free of dispersing agent.

2. The pharmaceutical composition of claim 1, wherein the pharmaceutically acceptable excipients comprises one or more of solubilizing agents, surfactants, and plasticizing agents.

3. The pharmaceutical composition of claim 1, wherein the composition comprises from about 5% to about 90% w/w of surfactant.

4. The pharmaceutical composition of claim 1, wherein the solubilizing agent comprise water, polyethylene glycol, or mixture thereof.

5. The pharmaceutical composition of claim 1, wherein the composition retains at least 90% w/w of total potency of diclofenac or salt thereof after storage at 40° C. and 75% relative humidity for at least 3 months.

6. A pharmaceutical composition of diclofenac or pharmaceutically acceptable salt thereof comprising one or more pharmaceutically acceptable; wherein the composition exhibits no significant difference in one or both of the rate and extent of absorption of diclofenac or salts thereof as compared to formulation of diclofenac marketed under the trade name Zipsor®, and characterized in that the composition is substantially free of dispersing agent.

7. A method of providing relief from mild to moderate acute pain in a patient comprises of administering to said patient the pharmaceutical composition of claim 1.

8. A method of treating acute post-bunionectomy or post-osteotomy pain in a patient comprises of administering to said patient the pharmaceutical composition of claim 1.

9. A hard gelatin capsule or soft gelatin capsule filled with the pharmaceutical composition of claim 1.
